(19) United States
(12) Patent Application Publication
(10) Pub. No.: US 2020/0230283 A1
(43) Pub. Date: Jul. 23, 2020

(54) NANO-OXIDE/KAOLIN COMPOSITE HEMOSTATIC ANTIBACTERIAL MATERIAL, HEMOSTATIC HEALING-PROMOTING DRESSING AND PREPARATION METHOD THEREOF

(71) Applicant: CENTRAL SOUTH UNIVERSITY, Hunan (CN)

(72) Inventors: Huaming YANG, Hunan (CN); Mei LONG, Hunan (CN); Yi ZHANG, Hunan (CN); Jing OUYANG, Hunan (CN); Liangjie FU, Hunan (CN)

(73) Assignee: CENTRAL SOUTH UNIVERSITY, Hunan (CN)

(21) Appl. No.: 16/726,813

(22) Filed: Dec. 24, 2019

(30) Foreign Application Priority Data
Jan. 23, 2019 (CN) 201910062718.6

(51) Int. Cl. A61L 24/00; A61L 24/02; A61L 26/00

(52) U.S. Cl. CPC A61L 24/0068; A61L 2400/04; A61L 24/0015; A61L 26/0066; A61L 26/008; A61L 26/0095; A61L 26/0004; A61L 2300/62; A61L 2300/404; A61L 2300/102; A61L 2300/10; A61L 2300/412; A61L 2300/45; A61L 2300/418; A61L 24/02

(57) ABSTRACT

The present invention belongs to the field of medical materials. A nano-oxide/kaolin composite hemostatic antibacterial material includes an iron oxide/kaolin composite carrier, and zinc oxide supported on the surface of the composite carrier. The present invention further provides the preparation and application of the composite hemostatic antibacterial material. Furthermore, the present invention provides a hemostatic healing-promoting dressing including the composite hemostatic antibacterial material disclosed by the present invention. The present invention surprisingly finds from research that the zinc oxide and iron oxide/kaolin composite carrier have a synergistic effect, and further cooperated with a special loading morphology, the synergistic effect of the two is unexpectedly enhanced, the hemostatic property and antibacterial property of the material are effectively improved, and moreover, the rate of wound healing is further improved.

### CROSS-REFERENCE TO RELATED APPLICATION

This application claims the priority benefit of China application serial no. 201910062718.6, filed on Jan. 23, 2019. The entirety of the above-mentioned patent application is hereby incorporated by reference herein and made a part of this specification.

### BACKGROUND

### Technical Field

The present invention belongs to the technical field of biomedical dressings, and particularly relates to a nano-oxide/kaolin composite hemostatic antibacterial material with hemostatic, anti-inflammatory and healing-promoting functions, and a preparation method thereof.

### Description of Related Art

Excessive blood loss is one of the main causes of traumatic death, and uncontrollable infection of wounds can also endanger life, especially in wars, major natural disasters and field operations. Therefore, post-traumatic hemostasis and infection control are a key part of pre-hospital assistance. In addition to a massive hemorrhage during wound healing, a chronic inflammation is an important factor hindering wound healing. For wound infection, that is, the chronic inflammation caused by a large number of bacteria colonizing in the wound, the materials commonly used for wound healing mainly include organic substances, such as chitosan, sodium alginate and novel high polymers; inorganic nano antibacterial material, such as nano silver, zinc oxide, cerium oxide, and the like. Based on the materials' own activity (electricity, adsorption, antibacterial performance), the materials directly act on the wound, or using organic polymer or inorganic nanomaterial as a carrier, functional substances such as antibacterial agents or growth factors are supported to promote wound healing.

Wound healing is a complex and ordered biological process, including a hemostasis and inflammation phase, a proliferative phase, and a maturity phase. After a trauma occurs, an inflammatory response occurs within a few hours, and thrombus provides a skeleton for cell migration and platelet aggregation. However, most dressings for wound healing are mainly applied to a stage separately, such as a separate hemostatic agent and antibacterial agent. At present, only a few materials, such as chlorhexidine acetate-supported hyaluronic acid and polyethylene glycol hydrogel, have been reported to have both hemostatic property and wound healing-promoting property. An organic polymer is generally used as a carrier, and has the advantages of good moisturizing effect and anti-adhesion. The antibacterial effect is achieved by loading an antibacterial agent, but the hemostatic effect is poor, the price is high, and the preparation process is complicated, which is not conducive to large-scale production and use. At present, gauze is mainly used in large-scale hemostasis first-aid in the hospital, which has poor hemostatic effect and no antibacterial property.

### SUMMARY

In view of the deficiencies of the prior art, a first objective of the present invention is to provide a nano-oxide/kaolin composite hemostatic antibacterial material, aiming to provide a material which has fast hemostatic speed, bacteriostatic effect, and capability of promoting wound healing.

A second objective of the present invention is to provide a preparation method of the nano-oxide/kaolin composite hemostatic antibacterial material.

A third objective of the present invention is to provide application of the nano-oxide/kaolin composite hemostatic antibacterial material.

A fourth objective of the present invention is to provide a hemostatic healing-promoting dressing composited with the innovative nano-oxide/kaolin composite hemostatic antibacterial material of the present invention.

A fifth objective of the present invention is to provide a preparation method of a hemostatic healing-promoting fiber membrane (a preferred hemostatic healing-promoting dressing) containing a nano-oxide/kaolin composite.

A nano-oxide/kaolin composite hemostatic antibacterial material includes an iron oxide/kaolin composite carrier, and zinc oxide loaded on the surface of the composite carrier.

The present invention provides a composite hemostatic antibacterial material which uses an iron oxide/kaolin composite as a carrier with zinc oxide particles supported on the surface thereof. The present invention surprisingly discovers from research that the zinc oxide and iron oxide/kaolin composite carrier have a synergistic effect, and further cooperated with a special loading morphology, the synergistic effect of the two is unexpectedly enhanced, the hemostatic property and antibacterial property of the material are effectively improved, and moreover, the rate of wound healing is further improved.

Research of the present invention finds that the morphological characteristic that the zinc oxide is loaded on the surface of the composite carrier is the key to ensure good synergy between the zinc oxide and the composite carrier. Usually, the zinc oxide is mainly used as an antibacterial material. The inventors innovatively find that when the zinc oxide is supported on the surface of an iron oxide/kaolin composite carrier, the hemostatic property of the material is unexpectedly and synergistically enhanced; moreover, the composite hemostatic antibacterial material with the loading morphology can further improve the antibacterial property of the material and further promote wound healing; and the composite hemostatic antibacterial material can be used for the treatment of chronic inflammation.

Preferably, the composite carrier is a homogeneous mixed material of iron oxide and kaolin, or a core-shell material with kaolin as the core and iron oxide as the shell.

Further preferably, the composite carrier is the core-shell material (also referred to as kaolin@iron oxide material in the present invention). When the composite carrier is the core-shell material, the composite hemostatic antibacterial material of the present invention has kaolin as the core. The surface of the core is coated with an iron oxide intermediate layer, and iron oxide intermediate layer is supported with a zinc oxide outer layer. The inventors have found that the composite hemostatic antibacterial material of the multi-layer structure may further enhance the synergistic effect among the materials, and further improve the hemostatic, antibacterial and healing-promoting properties of the material.

According to researches, the innovative material of the present invention can further control particles of the material and granularity of the supported zinc oxide. Thus, the synergistic effect of the material can be further improved.

The composite hemostatic antibacterial material has a particle size of 200-1000 nm.

Preferably, the granularity of the zinc oxide is nanometer, more preferably 10-100 nm, preferably 10-70 nm. When the granularity of the zinc oxide supported on the surface of the composite carrier can be controlled at preferred 10-70 nm, the synergistic properties of the material in antibacterial performance, hemostasis and healing-promoting performance can be further enhanced.

In addition to the control of the morphology and the granularity described above, further control of the proportion of materials helps to further enhance the synergistic effect of the material.

In the composite hemostatic antibacterial material, the weight percentage of the zinc oxide is 10%-50%, further preferably 20-30%. The weight percentage of the iron oxide is 20-40%. With the preferred proportion, the synergistic effect of the material in hemostasis and antibacterial performance may be further enhanced.

The present invention provides a preparation method of the nano-oxide/kaolin composite hemostatic antibacterial material, including: forming zinc hydroxide in situ on the surface of iron oxide/kaolin composite carrier by a precipitation method (in situ deposition), and then performing calcination treatment.

By the preparation method of the present invention, the iron oxide/kaolin composite carrier may induce in situ deposition of zinc hydroxide on the surface thereof, and the method of inducing in situ formation is the key to exerting the synergistic effect of the material. By the preparation method of the present invention, the composite hemostatic antibacterial material with excellent synergistic effect may be obtained.

The Iron oxide/kaolin composite carrier of the present invention may be prepared by an existing method. For example, when the composite carrier is the homogeneous mixed material of iron oxide and kaolin, the composite carrier may be obtained by physically mixing iron oxide with kaolin by, for example, ball milling. When the composite carrier is the core-shell material with kaolin as the core and iron oxide as the shell, iron hydroxide may be formed on the surface of kaolin by a precipitation method, followed by calcination.

In the preparation method of the present invention, the composite carrier is the kaolin@iron oxide material, which helps to further improve the in situ formation effect of zinc hydroxide, and helps to further improve the hemostatic and antibacterial properties of the prepared material.

A Zn source (zinc source) of the present invention is a water-soluble compound capable of providing Zn²⁺, and is preferably at least one of zinc acetate, zinc nitrate, zinc sulfate and the like.

In the present invention, the iron oxide/kaolin composite carrier is dispersed in water, the Zn source is added, and the pH of the system is adjusted to 10-11; and a precipitation reaction is performed to deposit hydroxide in situ on the surface of the composite carrier.

The zinc source may be added in the form of a solution. The concentration of zinc ions in the zinc source solution is 0.01-0.1 M (mol/L), preferably 0.03-0.05 M.

Preferably, the concentration of zinc ions in a precipitation starting solution is not less than 0.001 mol/L, further preferably controlled to be 0.001-0.015 M, more preferably 0.008-0.015 M. The adsorption concentration of the zinc ions on the surface of the carrier may be controlled by controlling the droplet velocity. With control at the concentration, the synergistic property of the obtained composite material, such as antibacterial property and hemostatic property, is improved more remarkably.

Preferably, the temperature of the deposition reaction is 30-40° C.

Preferably, the calcination temperature is 250-550° C.

Preferably, the calcination time is 2-4 h.

The present invention provides application of the nano-oxide/kaolin composite hemostatic antibacterial material in preparation of an external preparation having at least one of hemostasis, antibacterial, and wound healing promotion functions.

In the application of the present invention, a pharmaceutically effective amount of the composite hemostatic antibacterial material of the present invention is composited on a pharmaceutically acceptable medical carrier suitable for use in a bleeding point of trauma, to prepare the external preparation.

In the application, the composite hemostatic antibacterial material of the present invention may be used as an active ingredient to prepare the external preparation together with a pharmaceutically acceptable excipient.

Preferably, the external preparation is at least one of an external powder preparation, paint or dressing.

The present invention further provides a hemostatic healing-promoting dressing, including a dressing substrate, and the nano-oxide/kaolin composite hemostatic antibacterial material supported on the dressing substrate.

Preferably, the dressing substrate is at least one of a polymer fiber membrane and a hydrogel.

In the hemostatic healing-promoting dressing, the amount of the nano-oxide/kaolin composite hemostatic antibacterial material is not less than the pharmaceutically effective amount thereof.

Further preferably, the dressing substrate is a polymer fiber membrane. Research of the present invention finds that using the polymer fiber membrane to load the nano-oxide/kaolin composite hemostatic antibacterial material of the present invention may further synergistically enhance the healing effect; and the hemostatic healing-promoting dressing may be applied to control of acute massive bleeding and wound infection in the field.

The dressing substrate is a polymer fiber membrane, and the nano-oxide/kaolin composite hemostatic antibacterial material may be embedded in the fiber membrane, or exposed on the surface of the fiber membrane.

Further preferably, in the hemostatic antibacterial polymer fiber membrane, the weight content of the nano-oxide/kaolin composite hemostatic antibacterial material is 5%-20%.

The present invention further provides a preparation method of the hemostatic healing-promoting fiber membrane containing a nano-oxide/kaolin composite, including: dissolving polycaprolactone and gelatin in a solvent, and adding the nano-oxide/kaolin composite hemostatic antibacterial material; performing uniform mixing to obtain an electrostatic spinning solution; and performing electrostatic spinning on the electrostatic spinning solution to obtain the hemostatic healing-promoting fiber membrane containing the nano-oxide/kaolin composite.

In the present invention, by innovative electrostatic spinning, the innovative nano-oxide/kaolin composite hemostatic antibacterial material of the present invention is supported onto a membranous layer material of polycaprolactone and gelatin, which helps to improve the healing-promoting property of the material.

In the present invention, in the hemostatic healing-promoting fiber membrane, the nano-oxide/kaolin composite hemostatic antibacterial material of the present invention may be embedded in a polymer substrate, or exposed on the surface of the polymer substrate.

Preferably, in an electrostatic spinning process, the voltage of a tip and a collector is 15-25.0 kV, the distance is 15-30 cm, and the pushing speed is 0.003-0.01 mm/s.

The present invention further provides a preferred preparation method of the hemostatic healing-promoting fiber membrane containing the nano-oxide/kaolin composite, including the following steps:

1) adding and dispersing an iron oxide/kaolin composite in deionized water, dropwise adding a zinc acetate solution, heating the system to 40° C. after dropwise addition, dropwise adding a 5 wt % aqueous ammonia solution, and continuing the reaction for 1 h; separating and drying, calcining at 250° C.-550° C. for 2 h; then obtaining a zinc oxide supported iron oxide/kaolin composite;

2) adding polycaprolactone and gelatin in a mass ratio of 1:1 to a trifluoroethanol solution, performing stirring for 24 h, dropwise adding an acetic acid solution containing 0.2 v/v % trifluoroethanol, and continuing stirring; adding the zinc oxide-supported iron oxide/kaolin composite, and performing thorough stirring for 12-24 h to obtain an electrostatic spinning solution;

3) adopting an electrostatic spinning method, charging the mixed liquid into a 5 mL syringe, where the voltage of the tip and the collector is 15-25.0 kV, the distance is 15-30 cm, and the pushing speed is 0.003-0.01 mm/s; fixing gauze on a cylindrical receiver for collection, and adding a total of 15 mL of the mixed liquid for performing electrostatic spinning to obtain a zinc oxide/iron oxide/kaolin composite fiber membrane.

Preferably, the concentration of the zinc acetate solution in step 1) is 0.03 mol/L-0.05 mol/L.

Preferably, the solid-liquid ratio of polycaprolactone and gelatin respectively to trifluoroethanol in step 2) is 5%-10%.

Preferably, the addition amount of the zinc oxide-supported iron oxide/kaolin composite in step 2) is 5%-15% (based on the total weight of polycaprolactone and gelatin).

Preferably, the voltage of the tip and the collector is 20 kV.

The hemostatic healing-promoting fiber membrane containing the nano-oxide/kaolin composite of the present invention is prepared by an electrostatic spinning method, where the iron oxide/kaolin composite supported with zinc oxide is prepared first by using nano-iron oxide/kaolin as the carrier through a precipitation method; then, the spinning solution is prepared by mixing the zinc oxide-supported iron oxide/kaolin composite with polycaprolactone, gelatin and trifluoroethanol; and finally a nano-oxide/kaolin composite fiber membrane is obtained by the electrostatic spinning method.

Compared with the prior art, the technical solution of the present invention has the following advantages.

(1) The nano-oxide/kaolin composite hemostatic antibacterial material with the components and morphology of the present invention may synergistically enhance respective properties, enhances the hemostatic effect, and has the advantages of being rapidly hemostatic, antibacterial and anti-inflammation, and promoting wound healing.

(2) The hemostatic healing-promoting dressing of the present invention is especially the hemostatic healing-promoting fiber membrane containing the nano-oxide/kaolin composite, and may further improve the advantages of the innovative nano-oxide/kaolin composite hemostatic antibacterial material of the present invention in hemostasis and wound healing; and moreover, the hemostatic healing-promoting dressing has no powder residue during use, is convenient for wound cleaning and convenient to use, and is especially suitable for hemostasis and wound healing in fields.

(3) The material of the present invention has the advantages of being wide and abundant in sources of raw materials and low in cost.

(4) The preparation method of the material of the present invention is simple in steps, easy to operate, and advantageous for large-scale production.

### BRIEF DESCRIPTION OF THE DRAWINGS

FIG. 1 is a scanning electron micrograph of an iron oxide/kaolin composite with nano zinc oxide supported on the surface (labeled as ZnO/Kaolin@Fe₂O₃-3) prepared in Embodiment 4.

FIG. 2 is a transmission electron micrograph of the iron oxide/kaolin composite with nano zinc oxide supported on the surface (labeled as ZnO/Kaolin@Fe₂O₃-3) prepared in Embodiment 4.

FIG. 3 is a physical photograph of a hemostatic healing-promoting fiber membrane of ZnO/Kaolin@Fe₂O₃-3 (labeled as ZnO-Fe₂O₃-Kaolin-3/PG) prepared in Embodiment 5.

FIG. 4 is a scanning electron micrograph of the hemostatic healing-promoting fiber membrane of ZnO/Kaolin@Fe₂O₃-3 (labeled as ZnO/Kaolin@Fe₂O₃-3/PG) prepared in Embodiment 5.

### DESCRIPTION OF THE EMBODIMENTS

To make the objectives, technical solutions and advantages of the present invention clearer, the present invention will be further described in detail below with reference to accompanying drawings and embodiments. It should be understood that the specific embodiments described herein are merely illustrative of the present invention and are not intended to limit the present invention.

A hemostatic antibacterial material containing a zinc oxide/Fe₂O_{3/}nano-kaolin composite of the present invention may be used for bleeding wounds to accelerate hemostasis rate, resist bacterial infection of wounds, and promote wound healing.

A chemical formula of a term “kaolin” in the present specification is Al₂SiO₂.2H₂₀. In some forms, kaolin contains about 45.31% of silica, about 37.21% of alumina, and about 14.1% of water.

The nano-kaolin in the embodiments in the present specification is a standard product of China Kaolin Clay Co., Ltd.

### Embodiment 1

The present embodiment provides a preparation method of an Fe₂O₃/nano-kaolin composite, and the method includes the following steps.

0.6 g of sodium hydroxide was weighed, and 150 mL of water was added to prepare a 0.1 mol/L sodium hydroxide solution. 2.7 g of FeCl₃.6H₂₀ was weighed, and 100 mL of deionized water was added to prepare a 0.1 mol/L FeCl₃ solution. 150 mL of a 0.1 mol/L NaOH solution was slowly added dropwise to 100 mL of the 0.1 mol/L FeCl₃ solution in a water bath of 70° C. under vigorous stirring. After dropwise addition, the mixed liquid was taken out to cool down slowly to obtain a stable reddish-brown transparent iron polymer solution for use. The polymer solution was labeled as a polymeric hydroxy iron ion solution.

1 g of nano-kaolin was weighed and added to 50 mL of the 0.1 mol/L polymeric hydroxy iron ion solution. The pH of a reaction system was adjusted with a 5 mol/L NaOH solution to 3. The reaction system was heated in the water bath to 60° C. and subjected to magnetic stirring for 5 h. A product was washed, separated and dried at 60° C. The product was calcined in an air atmosphere at 250° C. for 1 h, 350° C. for 1 h, and 550° C. for 4 h respectively. A composite carrier coated with Fe₂O₃ on the surface of kaolin was obtained and labeled as Kaolin@Fe₂O₃ (iron oxide-coated nano-kaolin composite), and a granularity was 200-1000 nm.

### Embodiment 2

0.5 g of Kaolin@Fe₂O₃ (prepared in Embodiment 1) was weighed, 130 mL of deionized water was added, and the mixed liquid was thoroughly stirred and subjected to ultrasonic dispersion to prepare a suspension. 50 mL of a 0.01 mol/L Zn(Ac)₂.2H₂O solution was dropwise added under vigorous stirring, so concentration of Zn²⁺ in a precipitation starting solution was 0.003 mol/L. After the temperature of a reaction system was increased to 40° C., 20 mL of a 5 wt % aqueous ammonia solution was dropwise added (the pH was maintained at 10-11 in a precipitation process), and the reaction lasted for 1 h. After reaction, a product was centrifugally separated and washed with deionized water for three times. After being dried at 60° C., the product was calcined at 250° C. (in an air atmosphere) for 2 h to prepare the material with nano zinc oxide supported on a Kaolin@Fe₂O₃ carrier, the material was labeled as ZnO/Kaolin@Fe₂O₃-1, the granularity of the material was 200-1000 nm, and the particle size of the surface supported zinc oxide was about 40 nm.

The prepared ZnO/Kaolin@Fe₂O₃-1 was sealed and stored in a drying vessel in which allochroic silicagel was placed at the bottom for use.

### Embodiment 3

Compared with Embodiment 2, the main difference is that the concentration of Zn²⁺ in the precipitation starting solution was increased to 0.008 mol/L, and the specific operations are as follows:

0.5 g of Kaolin@Fe₂O₃ (prepared in Embodiment 1) was weighed, 130 mL of deionized water was added, and the mixed liquid was thoroughly stirred and subjected to ultrasonic dispersion to prepare a suspension. 50 mL of a 0.03 mol/L Zn(Ac)2.2H20 solution was dropwise added under vigorous stirring. After the temperature of the reaction system was increased to 40° C., 20 mL of a 5 wt % aqueous ammonia solution was dropwise added, and the reaction lasted for 1 h. After reaction, a product was centrifugally separated and washed with deionized water for three times. After being dried at 60° C., the product was calcined at 250° C. for 2 h to prepare a Kaolin@Fe₂O₃ material with nano zinc oxide supported on the surface, the Kaolin@Fe₂O₃ material was labeled as ZnO/Kaolin@Fe₂O₃-2, the granularity of the material was 200-1000 nm, and the particle size of the surface supported zinc oxide was about 70 nm.

### Embodiment 4

Compared with Embodiment 2, the main difference is that the concentration of zinc ions of a zinc source added to the reaction system was increased, the concentration of Zn²⁺ in the added zinc source solution was 0.014 mol/L, and the specific operations are as follows:

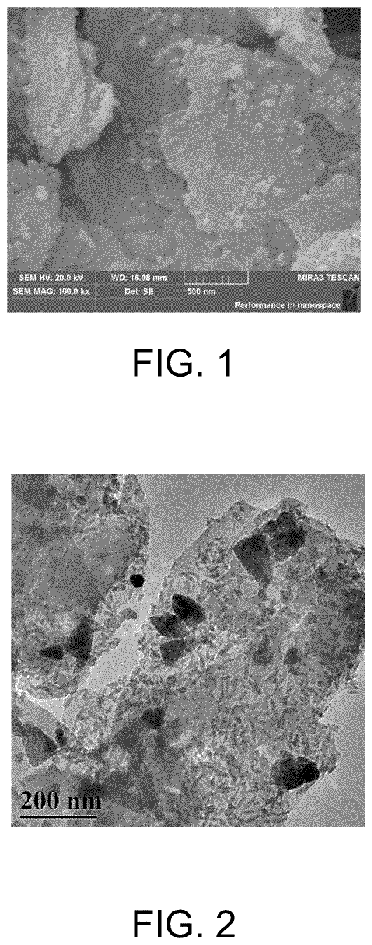

0.5 g of Kaolin@Fe₂O₃ (prepared in Embodiment 1) was weighed, 130 mL of deionized water was added, and the mixed liquid was thoroughly stirred and subjected to ultrasonic dispersion to prepare a suspension. 50 mL of a 0.05 mol/L Zn(Ac)2.2H20 solution was dropwise added under vigorous stirring. After the temperature of the reaction system was increased to 40° C., 20 mL of a 5 wt % aqueous ammonia solution was dropwise added, and the reaction lasted for 1 h. After reaction, the product was centrifugally separated and washed with deionized water for three times. After being dried at 60° C., a product was calcined at 250° C. for 2 h and sealed and stored in a drying vessel in which allochroic silicagel was placed at the bottom for use. The material was labeled as ZnO/Kaolin@Fe₂O₃-3, the granularity of the material was 200-1000 nm, and the particle size of the surface supported zinc oxide was about 100 nm. The SEM graph of the material is shown in FIG. 1. The TEM graph of the material is shown in FIG. 2. FIG. 1 and FIG. 2 show the morphological patterns of zinc oxide-iron oxide-kaolin-3. Iron oxide with small particles is coated on kaolin. The larger particles are zinc oxide dispersed and supported on iron oxide-kaolin.

### Embodiment 5

The present embodiment provides preparation of a zinc oxide/Fe₂O₃/nano kaolin-polycaprolactone/gelatin electrostatic spinning membrane (hemostatic healing-promoting fiber membrane), and the method includes the following steps.

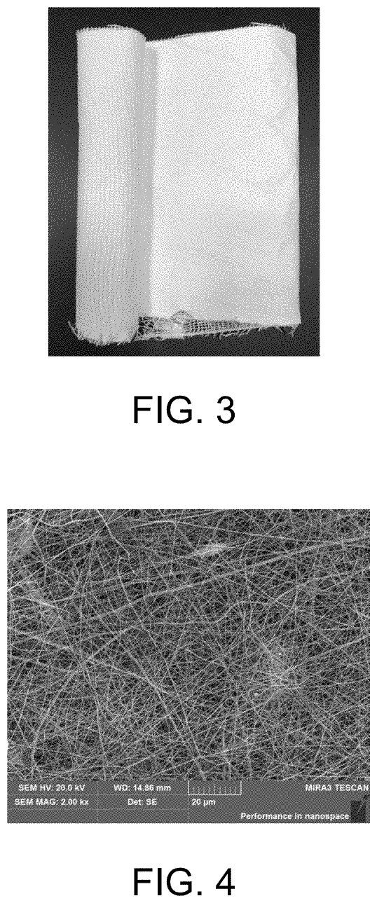

1 g of gelatin (type A, 300 bloom, prepared by an acid method) was weighed, 15 mL of trifluoroethanol was added, the mixed liquid was stirred and dissolved, 1 g of polycaprolactone (Mn=80000, Sigma-Aldrich) was added, and the mixed liquid was stirred at room temperature for 12 h. An acetic acid solution containing 0.2 v/v % trifluoroethanol was dropwise added. After uniform stirring, 0.13 g of a ZnO/Kaolin@Fe₂O₃-3 composite was added, and the mixed liquid was stirred continuously for 12 h to uniformly disperse the composite. The mixed liquid was charged into a 5 mL syringe, and a membrane was prepared by using an electrostatic spinning device, where the voltage of the tip and the collector was 20.0 kV, the distance was 15 cm, and the pushing speed was 0.003 mm/s. Gauze was fixed on a cylindrical receiver for collection, and a total of 15 mL of the mixed liquid was added for performing electrostatic spinning. After drying was performed at room temperature for 0.5 h, a fiber membrane with ZnO/Kaolin@Fe₂O₃-3 composited on the surface was obtained and labeled as ZnO/Kaolin@Fe₂O₃-3/PG. The physical image of the material is shown in FIG. 3, and the SEM graph is shown in FIG. 4.

### Comparative Example 1

Compared with Embodiment 1, the difference is that no oxide (iron oxide and zinc oxide) was added, and the conditions of calcination in Embodiment 1 were used for treatment, specifically as follows:

3 g of nano-kaolin was weighed and calcined in an air atmosphere at 250° C. for 1 h, 350° C. for 1 h, and 550° C. for 4 h respectively. The product was sealed and stored in a drying vessel in which allochroic silicagel was placed at the bottom for use. The product was labeled as Kaolin_{H}.

### Comparative Example 2

Compared with Embodiment 5, the difference is that the nano-oxide/kaolin composite hemostatic antibacterial material (ZnO/Kaolin@Fe₂O₃-3) of the present invention was not added, specifically as follows:

1 g of gelatin (type A, 300 bloom, prepared by an acid method) was weighed, 15 mL of trifluoroethanol was added, the mixed liquid was stirred and dissolved, 1 g of polycaprolactone

(Mn=80000, Sigma-Aldrich) was added, and the mixed liquid was stirred at room temperature for 12 h. An acetic acid solution containing 0.2 v/v % trifluoroethanol was dropwise added. After uniform stirring, the mixed liquid was stirred continuously for 12 h. The mixed liquid was charged into a 5 mL syringe, and a membrane was prepared using an electrostatic spinning device, where the voltage of the tip and the collector was 20.0 kV, the distance was 15 cm, and the pushing speed was 0.003 mm/s. Gauze was fixed on a cylindrical receiver for collection, and a total of 15 mL of the mixed liquid was added for performing electrostatic spinning. After drying was performed at room temperature for 0.5 h, the product was sealed and stored in a drying vessel in which allochroic silicagel was placed at the bottom for use. The product was labeled as PG.

### Application Embodiment

Antibacterial Experiment

A colony counting method was used, Escherichia coli (ATCC8739, Guangdong Institute of Microbiology) was used as an object, the bacterial concentration was 1×10^{5·6} CFU mL⁻¹, the concentration of antibacterial powder was 0.001 g/mL, and co-culture was performed for 3 h. The antibacterial results of the cases are shown in Table 1.

TABLE 1

| Case | Material | Bacterial survival rate (%) |
|---|---|---|
| Comparative | Kaolin_{H} | 113 ± 4.7 |
| example 1 | | |
| Embodiment 1 | Kaolin@Fe₂O₃ | 21.8 ± 4 |
| Embodiment 2 | ZnO/Kaolin@Fe₂O₃-1 | 9.4 ± 2.5 |
| Embodiment 3 | ZnO/Kaolin@Fe₂O₃-2 | 4.2 ± 0.9 |
| Embodiment 4 | ZnO/Kaolin@Fe₂O₃-3 | 0.4 ± 0.1 |
| Comparative | PG | No obvious antibacterial |
| example 2 | | property |

The bacterial survival rates of Kaolin_{H}, Kaolin@Fe₂O₃, ZnO/Kaolin@Fe₂O₃-1, ZnO/Kaolin@Fe₂O₃-2 and ZnO/Kaolin@Fe₂O₃-3 are 113±4.7%, 21.8±4%, 9.4±2.5%, 4.2±0.9% and 0.4±0.1% respectively. Under the conditions, the ZnO/Kaolin@Fe₂O₃-3 has the lowest bacterial survival rate, the bacteria are basically inhibited, and the composite has the best bacteriostatic effect.

Hemostasis Experiment

Male BALB/C mice weighing 18.0-22.0 g were randomly grouped by body weight. The mice were immobilized, the tails were revealed, and a 1 cm wound was cut with a surgical blade at the tail ends of the mice to bleed the mice. After the incision, the corresponding material powder (shown in Table 2) was immediately given, and the bleeding time was recorded with a timer. The hemostasis time of each case is shown in Table 2.

TABLE 2

| Case | Material | Bleeding time (s) |
|---|---|---|
| Blank control group | No material | 223 ± 66 |
| Comparative example 1 | Kaolin_{H} | 163 ± 33 |
| Embodiment 1 | Kaolin@Fe₂O₃ | 178 ± 57 |
| Embodiment 4 | ZnO/Kaolin@Fe₂O₃-3 | 129 ± 9 |
| Comparative example 2 | PG | 203 ± 93 |

From Table 2, the Kaolin@Fe₂O₃ supported with the nano zinc oxide on the surface may effectively improve the hemostasis rate.

Healing Experiment

Male BALB/C mice weighing 18.0-22.0 g were randomly grouped by body weight. Each group of mice was anesthetized by intraperitoneal injection of chloral hydrate (10%). A circular wound of 2 cm in diameter was cut on the back skin of the mice with a pair of scissors, and then 100 uL of Escherichia coli (1×10^{5·6} CFU mL⁻¹) was dropwise added. After 30 min, self-made adhesive bandages containing the material powder of Table 3 or the fiber membrane of Embodiment 5 was applied. The drug was administered every other day for 14 days. The wound area of each group of mice was measured respectively on the 3rd, 7th and 14th days after administration, and the bacterial concentration of the wound was detected. ZnO/Kaolin@Fe₂O₃-3/PG significantly increased the healing area of the mouse wounds on the third day of administration. ZnO/Kaolin@Fe₂O₃-3 and ZnO/Kaolin@Fe₂O₃-3/PG significantly reduced the quantity of bacteria in the mice wounds on the 7th and 14th day after administration. ZnO/Kaolin@Fe₂O₃-3 effectively inhibited the proliferation of wound bacteria and promoted wounds healing in a powder form or after being spun with polycaprolactone/gelatin (PG). The healing data is shown in Table 3.

TABLE 3

| | | Percentage of |
|---|---|---|
| | | healing area |
| Case | Material | after 14 days (%) |
| Blank control group | Medical gauze | 81.54 ± 4.94 |
| Comparative example 1 | Kaolin_{H} | 88.45 ± 1.92 |
| Embodiment 1 | Kaolin@Fe₂O₃ | 85.53 ± 1.9 |
| Embodiment 4 | ZnO/Kaolin@Fe₂O₃-3 | 88.49 ± 6.94 |
| Embodiment 5 | ZnO/Kaolin@Fe₂O₃-3/PG | 91.19 ± 2.25 |
| Comparative example 1 | PG | 88.31 ± 0.98 |

As shown in Table 3, if the hemostatic antibacterial material of the present invention is composited on a carrier (e.g., a fiber membrane), wound healing may be promoted.

The above description is only preferred embodiments of the present invention and is not intended to limit the present invention. Any modifications, equivalent substitutions, improvements and the like made within the spirit of the present invention are included within the scope of the invention.

What is claimed is:

1. A nano-oxide/kaolin composite hemostatic antibacterial material, comprising:
an iron oxide/kaolin composite carrier; and
zinc oxide supported on a surface of the iron oxide/kaolin composite carrier.

2. The nano-oxide/kaolin composite hemostatic antibacterial material according to claim 1, wherein the iron oxide/kaolin composite carrier is a homogeneous mixed material of iron oxide and kaolin, or a core-shell material with kaolin as a core and iron oxide as a shell.

3. The nano-oxide/kaolin composite hemostatic antibacterial material according to claim 1, wherein the nano-oxide/kaolin composite hemostatic antibacterial material has a particle size of 200-1000 nm; and the zinc oxide has a particle size of 10-100 nm.

4. The nano-oxide/kaolin composite hemostatic antibacterial material according to claim 1, wherein a weight percentage of the zinc oxide is 10%-50%; and a weight percentage of the iron oxide is 20-40%.

5. A preparation method of the nano-oxide/kaolin composite hemostatic antibacterial material according to claim 1, comprising forming zinc hydroxide in situ on the surface of the iron oxide/kaolin composite carrier by a precipitation method, and then performing calcination treatment.

6. The preparation method of the nano-oxide/kaolin composite hemostatic antibacterial material according to claim 5, further comprising:
dispersing the iron oxide/kaolin composite carrier in water, adding a Zn source, and adjusting pH of a system to 10-11; and
performing a precipitation reaction to deposit the zinc hydroxide in situ on the surface of the iron oxide/kaolin composite carrier.

7. The preparation method of the nano-oxide/kaolin composite hemostatic antibacterial material according to claim 6, wherein a concentration of zinc ions in a precipitation starting solution is not less than 0.001 mol/L.

8. The preparation method of the nano-oxide/kaolin composite hemostatic antibacterial material according to claim 7, wherein the concentration of zinc ions in the precipitation starting solution is 0.001-0.03 mol/L.

9. The preparation method of the nano-oxide/kaolin composite hemostatic antibacterial material according to claim 8, wherein the concentration of zinc ions in the precipitation starting solution is 0.008-0.015 mol/L.

10. The preparation method of the nano-oxide/kaolin composite hemostatic antibacterial material according to claim 6, wherein a temperature of the deposition reaction is 30-40° C.

11. The preparation method of the nano-oxide/kaolin composite hemostatic antibacterial material according to claim 6, wherein a calcination temperature is 250-550° C., and a calcination time is 2-4 h.

12. A preparation method of an external preparation having at least one of hemostatic, antibacterial, and wound healing promotion functions, comprising:
using the nano-oxide/kaolin composite hemostatic antibacterial material according to claim 1.

13. The preparation method of the external preparation having at least one of hemostatic, antibacterial, and wound healing promotion functions according to claim 12, wherein the external preparation is at least one of an external powder preparation, paint or dressing.

14. A preparation method of an external preparation having at least one of hemostatic, antibacterial, and wound healing promotion functions, comprising:
using the nano-oxide/kaolin composite hemostatic antibacterial material prepared by the preparation method according to claim 5.

15. A hemostatic healing-promoting dressing, comprising:
a dressing substrate; and
the nano-oxide/kaolin composite hemostatic antibacterial material according to claim 1, supported on the dressing substrate.

16. The hemostatic healing-promoting dressing according to claim 15, wherein the dressing substrate is at least one of a polymer fiber membrane and a hydrogel;
and a weight content of the nano-oxide/kaolin composite hemostatic antibacterial material is 5%-20%.

17. A hemostatic healing-promoting dressing, comprising:
a dressing substrate; and
the nano-oxide/kaolin composite hemostatic antibacterial material prepared by the preparation method according to claim 5, supported on the dressing substrate.

18. A preparation method of a hemostatic healing-promoting fiber membrane containing a nano-oxide/kaolin composite, comprising:
dissolving polycaprolactone and gelatin in a solvent;
adding the nano-oxide/kaolin composite hemostatic antibacterial material according to claim 1;
performing uniform mixing to obtain an electrostatic spinning solution; and
performing electrostatic spinning on the electrostatic spinning solution to obtain the hemostatic healing-promoting fiber membrane containing the nano-oxide/kaolin composite.

19. The preparation method of the hemostatic healing-promoting fiber membrane containing the nano-oxide/kaolin composite according to claim 18, wherein in an electrostatic spinning process, the voltage of a tip and a collector is 15-25.0 kV, the distance is 15-30 cm, and the pushing speed is 0.003-0.01 mm/s.

20. A preparation method of a hemostatic healing-promoting fiber membrane containing a nano-oxide/kaolin composite, comprising:
dissolving polycaprolactone and gelatin in a solvent;
adding the nano-oxide/kaolin composite hemostatic antibacterial material prepared by the preparation method according to claim 5;
performing uniform mixing to obtain an electrostatic spinning solution; and
performing electrostatic spinning on the electrostatic spinning solution to obtain the hemostatic healing-promoting fiber membrane containing the nano-oxide/kaolin composite.
